(19) United States
(12) Patent Application Publication
(10) Pub. No.: US 2025/0340945 A1
(43) Pub. Date: Nov. 6, 2025

(54) CIRCULATING TUMOR DNA AND METHODS OF USE THEREOF

(71) Applicant: H. LEE MOFFITT CANCER CENTER AND RESEARCH INSTITUTE, INC., Tampa, FL (US)

(72) Inventors: Frederick L. LOCKE, Lutz, FL (US); Erin DEAN, Tampa, FL (US)

(21) Appl. No.: 18/859,155

(22) PCT Filed: Apr. 25, 2023
(86) PCT No.: PCT/US2023/019786

§ 371 Date: Oct. 22, 2024

Related U.S. Application Data
(60) Provisional application No. 63/334,285, filed on Apr. 25, 2022.

(51) Int. Cl. C12Q 1/6886; A61K 40/11; A61K 40/31; A61K 40/42; A61P 35/00

(52) U.S. Cl. CPC C12Q 1/6886; A61K 40/11; A61K 40/31; A61K 40/4211; A61P 35/00; C12Q 2600/106; C12Q 2600/156

(57) ABSTRACT

The present disclosure relates circulating tumor DNA and methods of treating, preventing, and diagnosing cancer. The present disclosure also provides methods of assessing the efficacy of a therapy after cancer diagnosis.

### RELATED APPLICATION

This PCT application claims the benefit of, U.S. Provisional Patent Application No. 63/334,285, filed Apr. 25, 2022, which is incorporated by reference herein in its entirety.

### FIELD

The present disclosure relates circulating tumor DNA and methods of treating, preventing, and diagnosing cancer. The present disclosure also provides methods of assessing the efficacy of a therapy after cancer diagnosis.

### BACKGROUND

Current techniques including tumor biopsies and other invasive methods are common practices for providing patients with a diagnosis and for determining tumor burden in several cancers. However, additional approaches are needed to noninvasively, efficiently, and accurately identify tumors while also maximizing disease management and minimizing physical pain and emotional distress to the patient. Such approaches are also needed to diagnose and monitor disease progression as well as assess the efficacy of current treatments. The compositions and methods disclosed herein address the need for improved techniques of diagnosing, monitoring, and/or treating cancer before or after a prescribed treatment.

### SUMMARY

The present disclosure provides methods of treating, preventing, reducing, and/or diagnosing cancer in a subject. The present disclosure also provides a method of assessing the efficacy of a CAR T cell therapy in a subject diagnosed with cancer.

In one aspect, disclosed herein is a method of treating or preventing cancer in a subject in need thereof, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, wherein the presence of ctDNA and MTV in the subject or an increase in ctDNA and MTV in the subject relative to a control indicate the subject has a cancer, and administering a treatment to the subject.

In one aspect, disclosed herein is a method of diagnosing a cancer in a subject, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, wherein the presence of ctDNA and MTV in the subject or an increase in ctDNA and MTV in the subject relative to a control indicate the subject has a cancer.

In some embodiments, the ctDNA is assayed with a DNA sequencing method. In some embodiments, the DNA sequencing method comprises next generation sequencing (NGS). In some embodiments, the imaging modality comprises a positron emission tomography (PET) scan.

In some embodiments, the tissue sample comprises a plasma sample. In some embodiments, the treatment comprises a chimeric antigen receptor (CAR) T cell therapy. In some embodiments, the CAR T cell therapy comprises T cells that comprise a chimeric antigen receptor specific for CD19. In some embodiments, the CAR T cell therapy comprises an axicabtagene ciloleucel (axi-cel) therapy.

In some embodiments, the cancer comprises a first tumor, a relapse tumor, or a refractory tumor. Ins some embodiments, the cancer comprises a large B-cell lymphoma. In some embodiments, the subject has a relapse or refractory large B-cell lymphoma. In some embodiments, the method prevents recurrence of the cancer in the subject.

In one aspect, disclosed herein is a method of assessing efficacy of a chimeric antigen receptor (CAR) T cell therapy administered to a subject diagnosed with cancer, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, observing the subject for a level of inflammation, and quantifying a correlation between the ctDNA, the MTV, the level of inflammation, and a concentration of CAR T cells, wherein efficacy of the CAR T cell therapy is ineffective when the ctDNA and MTV increases or remains unchanged relative to the concentration of CAR T cells, and wherein efficacy of the CAR T cell therapy is effective when ctDNA and MTV decreases relative to the concentration of CAR T cells.

In some embodiments, the correlation is quantified over a period of time.

In some embodiments, the method quantifies a rate of ctDNA degradation. In some embodiments, the method quantifies a rate of tumor volume reduction, a rate of tumor volume growth, and a rate of tumor killing.

In some embodiments, the rate of tumor killing is proportionate to the concentration of CAR T cells. In some embodiments, the method quantifies a rate of ctDNA moving from a cancer cell into peripheral blood. In some embodiments, the method quantifies a rate of inflammation clearance.

In some embodiments, efficacy is assessed at least 30 days after the subject is administered the CAR T cell therapy. In some embodiments, efficacy is assessed 30 days after the subject is administered the CAR T cell therapy. In some embodiments, efficacy is assessed 60 days after the subject is administered the CAR T cell therapy. In some embodiments, efficacy is assessed 90 days after the subject is administered the CAR T cell therapy.

In some embodiments, the ctDNA is measured one, two, or more times. In some embodiments, the MTV is measured one, two, or more times. In some embodiments, the level of inflammation is measured one, two, or more times.

In some embodiments, the method provides an updated assessment of the subject response to the CAR T cell therapy. In some embodiments, the method prevents recurrence of the cancer in the subject.

### BRIEF DESCRIPTION OF FIGURES

The accompanying figures, which are incorporated in and constitute a part of this specification, illustrate several aspects described below.

FIGS. 1A, 1B, 1C, 1D, 1E, and 1F show the correlation of MTV with ctDNA at baseline, 1 month, and 3 months. The correlation of baseline MTV with ctDNA strengthened in the Focused Cohort (FIG. 1B) compared to the Entire Cohort (FIG. 1A). The correlation was moderate at 1 month (FIG. 1C) and the strongest at 3 months (FIG. 1D). For patients with evidence of disease, there was no correlation at 1 month (FIG. 1E), but the correlation persisted at 3 months (FIG. 1F).

FIGS. 2A, 2B, 2C, and 2D show the correlation of baseline, 1 month, and 3 month MTV with ctDNA at different timepoints. FIGS. 2A and 2B show baseline MTV correlated best with ctDNA at pre-lymphodepletion (n=41) (rs 0.61, P<0.0001) within the first month and overall: day 0 (n=28) (rs 0.56, P=0.002), day 7 (n=41) (rs 0.57, P=0.0001), day 14 (n=38) (rs 0.6, P<0.0001), day 21 (n=33) (rs 0.54, P=0.001), day 28 (n=38) (rs 0.6, P<0.0001), day 56 (n=23) (rs 0.64, P=0.001), day 90 (n=31) (rs 0.48, P=0.006), day 180 (n=19) (rs 0.53, P=0.02), day 270 (n=5) (rs 0.71, P=0.4), and day 365 (n=13) (rs 0.58, P=0.04). FIG. 2C shows MTV at 1 month correlated best with ctDNA on day 28 (n=53) (rs 0.60, P<0.0001): pre-lymphodepletion (n=56) (rs 0.38, P=0.004), day 0 (n=43) (rs 0.50, P=0.0007), day 7 (n=56) (rs 0.58, P<0.0001), day 14 (n=53) (rs 0.50, P=0.0001), day 21 (n=48) (rs 0.59, P<0.0001), day 56 (n=34) (rs 0.41, P=0.02), day 90 (n=41) (rs 0.48, P=0.002), day 180 (n=25) (rs 0.39, P=0.05), day 270 (n=12) (rs 0.32, P=0.15), and day 365 (n=19) (rs 0.18, P=0.47). FIG. 2D shows MTV at 3 months correlated best with ctDNA on day 90 (n=37) (rs 0.89, P<0.0001): pre-lymphodepletion (n=46) (rs 0.28, P=0.06), on day 0 (n=32) (rs 0.39, P=0.03), day 7 (n=46) (rs 0.54, P=0.0001), day 14 (n=44) (rs 0.46, P=0.002), day 21 (n=39) (rs 0.49, P=0.002), day 28 (n=44) (rs 0.41, P=0.006), day 56 (n=30) (rs 0.69, P<0.0001), day 180 (n=25) (rs 0.53, P=0.007), day 270 (n=12) (rs 0.23, P=0.64), day 365 (n=20) (rs 0.15, P=0.54).

FIGS. 3A and 3B show the Kaplan-Meier survival curves and log-rank p-values in the Focused Cohort at baseline by risk group. (FIG. 3A) PFS (FIG. 3B) OS is worse in the high-risk group (high MTV/high ctDNA) compared to the intermediate-risk (high MTV/low ctDNA or low MTV/high ctDNA) or low-risk (low MTV/low ctDNA) groups. MTV cutoff>147.5 mL. ctDNA cutoff>100 Lg/mL.

FIG. 4 shows the responses to axi-cel shown over time. The response of the 57 patients is shown over time. Responses evolved initially with most becoming final by 3 months. The patients with positive ctDNA at 1 month are marked with a dash by the colored boxes and their outcome tracked. At 1 month, 3 patients in CR who remained in durable remission had detectable ctDNA, while 5/6 patients who progressed by month 6 had undetectable ctDNA. 6/7 patients initially in PR who achieved CR by 6 months had ctDNA 0 Lg/mL at 1 month. 1/2 patients initially in SD had 0 ctDNA at all available time points pre and post axi-cel. 14/17 PR/SD who had PD had detectable ctDNA at 1 month (1 was unavailable).

FIGS. 5A, 5B, 5C, 5D, 5E, and 5F show the dynamical Systems Model of ctDNA, tumor, and inflammation. FIG. 5A shows Qualitative schematic of the tumor burden (FIG. 5B), surrounded by inflammation (I), and ctDNA in peripheral blood. FIG. 5B shows a compartment model schematic. FIG. 5C shows model kinetics inferred from a MTV+/ctDNA+ patient undergoing CAR T-cell therapy with ctDNA measurements (dots). The curve represents the modeled (fitted) ctDNA compartment dynamics. FIG. 5D shows the inferred model kinetics of MTV (I+B, solid line) and tumor alone (B, dashed) of the same patient. MTV is measured from the PET scan at day 30 (dot). This modeling successfully captures disease progression, although delayed compared to the actual clinical progression due to tumor burden. FIGS. 5E and 5F show inferred model kinetics for a patient who was MTV+/ctDNA− at day 30 and after. Here, the MTV eventually consists entirely of inflammation and is still undetectable at day 365, in line with the cDNA kinetics and the endpoint of complete response. LoD: estimated level of detection of 1 mL.

FIG. 6 shows a graphical representation of patients with PET positive findings or detectable ctDNA at one month and three month post CAR-T infusion.

FIG. 7 shows the mathematical modeling of transient inflammation at DLBCL sites after CAR-T, informed by ctDNA, recapitulated late disease progression or long-term remission in cases with PET+ disease at one month after CAR-T therapy.

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

The terms “about” and “approximately” are defined as being “close to” as understood by one of ordinary skill in the art. In one non-limiting embodiment the terms are defined to be within 10%. In another non-limiting embodiment, the terms are defined to be within 5%. In still another non-limiting embodiment, the terms are defined to be within 1%.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “control” is an alternative subject or sample used in an experiment for comparison purposes. A control can be “positive” or “negative.”

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

A “chimeric antigen receptor” is an artificial T cell receptor used for immunotherapy. CAR are protein receptors that have been engineered to give T cells an enhanced ability to target a specific protein. CAR receptors are chimeric because the antigen binding and T cell activating functions have been combined into a single receptor.

The term “cancer” is used to address any neoplastic disease, and is not limited to epithelial neoplasms (surface and glandular cancers; such a squamous cancers or adenomas)). It is used here to describe both solid tumors and hematologic malignancies, including epithelial (surface and glandular) cancers, soft tissue, and bone sarcomas, angiomas, mesothelioma, melanoma, lymphomas, leukemias and myeloma.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially.

As used herein, “monitoring” refers to the actions of observing and checking the progress or quality of a treatment or procedure over a period of time. “Monitoring” also refers to observing the course of a disease or condition, such as a cancer, over a period of time.

“Quantify”, “quantifying”, “quantification”, and any other grammatical variations thereof refer to the process of acquiring numerical values to determine, express, or measure an amount of a substance or signal.

“Analyze”, “analyzing”, “analysis”, and any other grammatical variations thereof refer to the process of methodically examining and detailing the constitution, nature, or structure of a composition, compound, biological material, or process. “Analyze”, “analyzing”, “analysis”, and any other grammatical variations thereof can also refer to the study or understanding of the parts of a whole.

“Assess”, “assessing”, “assessment”, and any other grammatical variations thereof refer to the process of determining the importance, the size, the amount, the quality, the ability, and/or the value of a particular property or characteristic including, but not limited to efficacy, therapeutic effects, disease state or progression, and subject's health.

As used herein, “efficacy” refers to the ability of a composition, compound, therapeutic method, or any other prescribed medication to produce an effect, ideally the desired effect, to decrease, eliminate, reduce, or mitigate a disease or disorder.

As used herein, “correlation” refers to a relation existing between two or more phenomena, objects, or between mathematical or statistical variables which tend to vary, be associated, or occur together in a way not expected on the basis of chance alone.

A “rate” also referred to as “rate of change”, as used herein, describes how one quantity changes in relation to another quantity. For example, a change in distance relative to or over a specific period of time describes the rate of an object moving from one location to another. “Rate” also refers to a measure, quantity, or frequency, typically one quantity measured relative to another measure.

Methods of Diagnosing, Treating, and/or Preventing Cancer

The present disclosure provides methods of treating, preventing, reducing, and/or diagnosing cancer in a subject. The present disclosure also provides a method of assessing the efficacy of a CAR T cell therapy in a subject diagnosed with cancer.

Current methods of diagnosing, treating, and preventing cancer initially requires collection of a tissue biopsy, which can be an invasive and painful process for the subject. Once the tissue sample is collected and analyzed, medical practitioners can provide a diagnosis and treatment options for the subject. However, there are limited noninvasive options for diagnosing patients with cancer. There is a need to develop noninvasive and effective methods for diagnosing, treating, and/or preventing cancer.

Thus, in one aspect, disclosed herein is a method of diagnosing a cancer in a subject, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, wherein the presence of ctDNA and MTV in the subject or an increase in ctDNA and MTV in the subject relative to a control indicate the subject has a cancer.

In one aspect, disclosed herein is a method of treating or preventing cancer in a subject in need thereof, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, wherein the presence of ctDNA and MTV in the subject or an increase in ctDNA and MTV in the subject relative to a control indicate the subject has a cancer, and administering a treatment to the subject.

As used herein, a circulating tumor DNA (ctDNA) refers to tumor-derived fragmented DNA molecules, usually found in the blood that is not associated with non-cancerous cells, not to be confused with cell-free DNA. In some embodiments, one or more samples of ctDNA can be collected from the subject. In some embodiments, 1, 2, 3, 4, 5, or more sample of ctDNA can be collected from the subject.

As used herein, a metabolic tumor volume (MTV) refers to the metabolically active volume of a tumor segmented using an imaging modality including, but not limited to positron emission tomography (PET), ¹⁸F-fluorodeoxyglucose PET (FDG-PET) and PET/computerized tomography (PET/CT), useful in contributing to patient diagnoses, predicting patient outcomes, and assessing treatment response. It should be understood that a widely acceptable practice comprises calculating the volume of subcutaneous, solid, and non-solid tumors.

“DNA sequencing” refers to a general laboratory technique for determining the exact sequence of nucleotides, or nucleic acid bases, in a DNA molecule. The sequence of the nucleotides (often referred to as adenine (A), thymine (T), cytosine (C), and guanine (G)) encodes the biological information that cells use to develop and function.

In some embodiments, the ctDNA is assayed with a DNA sequencing method. In some embodiments, the DNA sequencing method comprises next generation sequencing (NGS). In some embodiments, the DNA sequencing method includes, but is not limited to sanger sequencing, capillary electrophoresis and fragment analysis, polymerase chain reaction (PCR), reverse transcriptase PCR (RT-PCR), real time PCR, and RT-PCR.

In some embodiments, the imaging modality comprises a positron emission tomography (PET) scan. In some embodiments, the tissue sample comprises a plasma sample. In some embodiments, the tissue sample comprises a blood sample, a saliva sample (buccal swab), a urine sample, or a fecal sample. In some embodiments, the ctDNA is isolated from the tissue sample. In some embodiments, the ctDNA is isolated from a plasma sample. In some embodiments, the ctDNA is isolated from a blood sample, a saliva sample (buccal swab), a urine sample, or a fecal sample.

As used herein, CAR T cell therapy” refers to T cells being harvested and engineered with chimeric antigen receptors to treat cancer. CARs, also known as chimeric immunoreceptors, chimeric T cell receptors, or artificial T cell receptors, are receptors proteins that have been engineered to give T cells an additional ability to target a specific antigen. These receptors are chimeric in that they combine both antigen-binding (e.g., a scFV or TCR region) and T cell activating functions into a single receptor. The premise of CAR T cell therapy is to modify T cells to recognize cancer cells in order to more effectively target and destroy said cancer cells. CAR T cells are commonly derived from the patient or from the T cells of a second healthy donor. CAR T cell therapy has shown promise in treating certain cancers including, but not limited to hematological cancers (e.g., lymphomas, leukemias, and myelomas). CAR T cell therapies can be optimized using approaches that consider factors and characteristics that contribute to therapeutic levels of CAR T cell expansion.

In some embodiments, the treatment comprises a chimeric antigen receptor (CAR) T cell therapy. In some embodiments, the CAR T cell therapy comprises T cells that comprise a chimeric antigen receptor specific for CD19. In some embodiments, the CAR T cell therapy comprises an axicabtagene ciloleucel (axi-cel) therapy. In some embodiments the CAR T cell therapy comprises idecabtagene vicleucel, lisocabtagene maraleucel, tisagenlecleucel, or brexucabtagene autoleucel.

In general, administration of CAR T cell therapies are discussed in U.S. patent application Ser. No. 16/865,369 (′369), which is herein incorporated by reference in its entirety for teachings concerning dosing and administration of CAR T cell therapies. It describes administering CAR T cell therapies by injection, inhalation, or through the digestive tract, such as orally. It also describes that target dosing of CAR T cells may be between about 1×10⁶ and about 2×10⁶ CAR positive viable T cells per kg body weight, with a maximum of 2×10⁸ CAR positive viable T cells.

In some embodiments, the treatment is administered before or concurrently with a chemotherapeutic agent. Exemplary chemotherapeutic agents include, but are not limited to, anti-estrogens (e.g. tamoxifen, raloxifene, and megestrol), LHRH agonists (e.g. goserelin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonucleotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, caminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, caminomycin, aminopterin, and hexamethyl melamine.

In some embodiments, the treatment is administered before or concurrently with an anti-cancer agent. Exemplary anti-cancer agents include, but are not limited to, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

In some embodiments, the treatment is administered before or concurrently with an anti-inflammatory agent. Exemplary anti-inflammatory agents including, but is not limited to aspirin, ibuprofen, ketoprofen, naproxen, steroids, glucocorticoids (including, but not limited to betamethasone, budesonide, dexamethasone, hydrocortisone, hydrocortisone acetate, methylprednisolone, prednisolone, prednisone, and triamcinolone), methotrexate, sulfasalazine, lefunomide, anti-Tumor Necrosis Factor (TNF) medications, cyclophosphamide, and mycophenolate.

In some embodiments, the cancer comprises a large B-cell lymphoma. In some embodiments, the cancer includes, but is not limited to, hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease).

In some embodiments, the subject has a relapse or refractory large B-cell lymphoma. In some embodiments, the subject has a cancer of any preceding aspect that has relapsed or is refractory.

As used herein, a “first tumor” refers to the first occurrence or appearance of a tumor in a subject who previously did not present with cancer.

As used herein, “relapse” or a “relapse tumor”, also known as a “recurrence”, refers to when cancer returns after a period of remission, or displaying minimal signs or symptoms of cancer. A cancer relapse or recurrence occurs when at least one cancer cell remains after therapeutic or surgical attempts to remove the cancer.

A “refractory tumor” refers to a cancer or tumor that does not respond to treatment, specifically the cancer displays signs of resistance during the beginning phases of treatment or becomes resistant during treatment.

In some embodiments, the cancer comprises a first tumor, a relapse tumor, or a refractory tumor. In some embodiments, the method prevents recurrence of the cancer in the subject.

In some embodiments, the subject is a human.

### Methods of Assessing Efficacy

In one aspect, disclosed herein is a method of assessing efficacy of a chimeric antigen receptor (CAR) T cell therapy administered to a subject diagnosed with cancer, the method comprising isolating a tissue sample from the subject, wherein the tissue sample comprises circulating tumor DNA (ctDNA), capturing an image from the subject with an imaging modality, assaying the ctDNA in the tissue sample, measuring metabolic tumor volume (MTV) from the image, observing the subject for a level of inflammation, and quantifying a correlation between the ctDNA, the MTV, the level of inflammation, and a concentration of CAR T cells, wherein efficacy of the CAR T cell therapy is ineffective when the ctDNA and MTV increases or remains unchanged relative to the concentration of CAR T cells, and wherein efficacy of the CAR T cell therapy is effective when ctDNA and MTV decreases relative to the concentration of CAR T cells.

In some embodiments, the correlation is quantified over a period of time. In some embodiments, the correlation is quantified over a period of time. In some embodiments, the correlation is quantified every week, every 2 weeks, every 3 weeks, every 4 weeks, or more. In some embodiments, the correlation is quantified every month, every 2 months, every 3 months, every 4 months, every 5 months, every 6 months, every 7 months, every 8 months, every 9 months, every 10 months, every 11 months, every 12 months, or more. In some embodiments, the correlation is quantified every year, every 2 years, every 3 years, every 4 years, every 5 years, or more.

In some embodiments, the method quantifies a rate of ctDNA degradation. In some embodiments, the method quantifies a rate of tumor volume reduction, a rate of tumor volume growth, and a rate of tumor killing.

In some embodiments, the rate of tumor killing is proportionate to the concentration of CAR T cells. In some embodiments, the method quantifies a rate of ctDNA moving from a cancer cell into peripheral blood.

Inflammation is one of many symptoms of cancer used as a determinant of cancer progression. In some embodiments, the inflammation can be systemic, or occurring throughout the body. In some embodiments, the inflammation can be localized to a specific tissue, at the tumor, or near the tumor. Symptoms of inflammation include, but are not limited to appetite loss, weight fluctuations (e.g., weight gain or weight loss), loss of mobility, swelling, fatigue, pain, and depression. In some embodiments, the method quantifies a rate of inflammation clearance.

In some embodiments, efficacy is assessed at least one day before the CAR T cell therapy is administered to establish a baseline measurement. In some embodiments, efficacy is assessed 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days or more before the CAR T cell therapy is administered.

In some embodiments, efficacy is assessed at least 30 days after the subject is administered the CAR T cell therapy. In some embodiments, efficacy is assessed 30 days after the subject is administered the CAR T cell therapy. In some embodiments, efficacy is assessed 60 days after the subject is administered the CAR T cell therapy. In some embodiments, efficacy is assessed 90 days after the subject is administered the CAR T cell therapy. In some embodiments, efficacy is assessed 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, or more days after the subject is administered the CAR T cell therapy.

In some embodiments, the ctDNA is measured one, two, or more times. In some embodiments, the ctDNA us measured 1, 2, 3, 4, 5, 6, 7, 8, 9, 10, or more times.

In some embodiments, the MTV is measured one, two, or more times. In some embodiments, the MTV is measured 1, 2, 3, 4, 5, 6, 7, 8, 9, 10, or more times.

In some embodiments, the level of inflammation is measured one, two, or more times. In some embodiments, the level of inflammation is measured 1, 2, 3, 4, 5, 6, 7, 8, 9, 10, or more times.

In some embodiments, the method provides an updated assessment of the subject response to the CAR T cell therapy. In some embodiments, the method prevents recurrence of the cancer in the subject.

In some embodiments, the ctDNA is assayed with a DNA sequencing method. In some embodiments, the DNA sequencing method comprises next generation sequencing (NGS). In some embodiments, the DNA sequencing method includes, but is not limited to sanger sequencing, capillary electrophoresis and fragment analysis, polymerase chain reaction (PCR), reverse transcriptase PCR (RT-PCR), real time PCR, and RT-PCR.

In some embodiments, the imaging modality comprises a positron emission tomography (PET) scan. In some embodiments, the tissue sample comprises a plasma sample. In some embodiments, the tissue sample comprises a blood sample, a saliva sample (buccal swab), a urine sample, or a fecal sample. In some embodiments, the ctDNA is isolated from the tissue sample. In some embodiments, the ctDNA is isolated from a plasma sample. In some embodiments, the ctDNA is isolated from a blood sample, a saliva sample (buccal swab), a urine sample, or a fecal sample.

In some embodiments, the cancer comprises a large B-cell lymphoma. In some embodiments, the subject has a relapse or refractory large B-cell lymphoma. In some embodiments, the subject has a cancer of any preceding aspect that has relapsed or is refractory. In some embodiments, the cancer comprises a first tumor, a relapse tumor, or a refractory tumor.

In some embodiments, the subject is a human.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Circulating Tumor DNA Adds Specificity to Pet Following Axicabtagene Ciloeucel in Large B-Cell Lymphoma

The meaning of metabolically active lesions was examined on 1 month restaging nuclear imaging of patients with relapsed/refractory (R/R) large B-cell lymphoma (LBCL) receiving axicabtagene ciloleucel (axi-cel) by assessing the relationship between total metabolic tumor volume (MTV) on positron emission tomography (PET) scans and circulating tumor DNA (ctDNA) in the plasma. In this prospective multicenter sample collection study, MTV was retrospectively calculated via commercial software at baseline, 1 and 3 months post chimeric antigen receptor (CAR) T-cell therapy; ctDNA was available pre and post axi-cel. Spearman correlation coefficient (rs) was used to study the relationship between the variables and a mathematical model was constructed to describe tumor dynamics 1 month post CAR T-cell therapy. The median time between baseline scan and axi-cel infusion was 33 (range, 1-137) days for all 57 patients. For 41 of the patients with imaging within 33 days of axi-cel or imaging before that time but no bridging therapy, the correlation at baseline became stronger (rs 0.61, P<0.0001) compared to all patients (rs 0.38, P=0.004). Excluding patients in complete remission with no measurable residual disease, ctDNA and MTV at 1 month did not correlate (rs 0.28, P=0.11), but did correlate at 3 months (rs 0.79, P=0.0007). Modeling of tumor dynamics, which incorporated ctDNA and inflammation as part of MTV, recapitulated outcomes of patients with positive radiologic 1-month scans. These results showed that non-progressing hypermetabolic lesions on 1 month PET represent ongoing treatment response and their composition may be elucidated by concurrent ctDNA.

### Introduction

Treatment with CD19 targeted chimeric antigen receptor (CAR) T-cell therapy has led to unprecedented response rates in patients with relapsed or refractory (R/R) large B cell lymphomas (LBCL). Response determination 1 month post CAR T-cell therapy is critical to guide clinical care. However, interpretation of fluorine-18 fluorodeoxyglucose positron emission tomography/computed tomography (¹⁸F-FDG PET/CT) scan imaging can be challenging due to the lack of specificity of FDG-avid lesions, which may represent tumor, infection, and/or inflammation. While tissue biopsy remains the gold standard for assessment, there is a strong desire to identify tumor noninvasively, efficiently, and accurately to minimize patients' physical pain and emotional distress and expedite disease management.

It has previously been demonstrated that metabolic tumor volume (MTV) and circulating tumor DNA (ctDNA), although based on different principles, each can serve to identify and quantify tumor non-invasively and provide for prognostication of clinical response after CAR T-cell therapy.

It was contemplated that inflammation generated by continued CAR T-cell anti-tumor activity contributes to FDG-avidity on ¹⁸F-FDG PET/CT imaging and confounds standard radiological evaluation of response per the Lugano classification, particularly within a few months of CAR T-cell therapy administration. The primary objective of this retrospective study was to examine the relationship between MTV and ctDNA pre and post axicabtagene ciloleucel (axi-cel) in patients with R/R LBCL in order to elucidate the etiology of non-progressing lesions with FDG uptake of unclear significance on ¹⁸F-FDG PET/CT. The secondary goal was to describe mathematically the ongoing treatment response to axi-cel following infusion and evaluate if inflammation may factor into the 1 month imaging assessment.

### Methods

### Patients

Seventy-two patients were originally enrolled in a prospective multi-institutional study that assessed the role of ctDNA in prognostication before and after standard-of-care axi-cel in patients with R/R LBCL treated from February 2018 to June 2019 at Stanford University, Moffitt Cancer Center, and University of Maryland Medical Center. Based on baseline ¹⁸F-FDG PET/CT scan availability, 57 patients were included in the current retrospective study to form the Entire Cohort. To account for baseline MTV most closely representing disease at the time of treatment, patients who received bridging therapy with baseline ¹⁸F-FDG PET/CT imaging beyond the median time measured from the time of PET-CT imaging to infusion of axi-cel were excluded to create a Focused Cohort (n=41). Bridging therapy was defined as any therapy in-between apheresis and axi-cel infusion used to control lymphoma. Clinical data was collected retrospectively. No biopsies were performed of FDG-avid lesions in patients that had not already exhibited frank progression per Lugano response criteria. Approval for review of patient records was obtained from each center's Institutional Review Board.

### Tumor Burden Derivation

MTV in mL, with threshold>41% maximum standardized uptake value, was calculated retrospectively in a step-wise process on ¹⁸F-FDG PET/CT performed prior to, and at 1 and 3 months post axi-cel using custom tools on MIM PACS version 6.8.4 (MIM Software Inc, Cleveland, OH). The processing time per scan was recorded in minutes (min). Baseline MTV results for 15 of the patients had been derived. Official radiology responses per Lugano criteria were available for all patients who had a month 1 and 3 restaging scan.

ctDNA values in Lymphoma genomes per mL of plasma (Lg/mL) were previously derived via next-generation sequencing (NGS) from plasma in a CFD tube (Roche Diagnostics, Indianapolis, IN) pre-lymphodepletion (baseline) and post axi-cel. The clonotype found at the highest concentration was tracked after first being identified via PCR amplification of rearranged IgH-VDJ, IgH-DJ, and Igkappa/lambda regions using universal consensus primers from archival formalin-fixed paraffin-embedded samples or from the initial plasma sample collected. MRD sensitivity threshold was 10⁻⁶.

### Mathematical Modeling

The dynamics and interactions among normal T cells (N), CAR T cells (C), and the cancer (B) were described mathematically:

$$\frac{dN}{dt} = - {r}_{N} N ln [ \frac{N + C}{{K}_{N}} ] . \qquad ( 1 )$$

$$\frac{dC}{dt} = - {r}_{C} ( T ) C ln [ \frac{N + C}{{K}_{C}} ] . \qquad ( 2 )$$

$$\frac{dB}{dt} = ( {λ}_{B} - {δ}_{B} ) B - γ ( C ) B . \qquad ( 3 )$$

Here, T=N+C is the total lymphocyte count, and

$${r}_{C} ( T ) = {λ}_{C} + \frac{{b ( T - {K}_{N} )}^{2}}{{aT}^{2} + {( T - {K}_{N} )}^{2}} ,$$

where λ_{c} is a background expansion and the second term reflects that growth can be attenuated when the overall (largely normal) T cell population reaches capacity, modulated by the two parameters a and b. This model was extended to incorporate ctDNA (Z) and the assumption that MTV (V) comprises tumor (B) and inflammation (I) with relation: V=B+I, with the dynamics:

$$\frac{dZ}{dt} = θ [ {αδ}_{B} + βγ ( C ) ] B - {δ}_{Z} Z , \qquad ( 4 )$$

$$\frac{dI}{dT} = ϕγ ( C ) B - τ I . \qquad ( 5 )$$

To parameterize the mechanistic model, both previously established parameter values were used, as well as patient-level data from the Focused Cohort for V (MTV) and Z (ctDNA) at all available time points (described below) for two representative patients. ctDNA (Z) was assumed to enter peripheral blood when the tumor dies, consistent with empirical observations. The variable δ_{z} is the rate of degradation of ctDNA, which is assumed to be constant. The tumor B grows autonomously with birth rate λ_{B}, dies with rate δ_{B}, and experiences tumor killing at rate γ_{B}, proportional to the number of CAR T cells. The parameter that modulates ctDNA includes θ with units (Lg/mL)/(mL) which can be thought of as the average ctDNA moved into peripheral blood per mL of tumor. The parameters α,β are dimensionless and reflect the probability of ctDNA being released through cell death. ϕ represents the degree of inflammation caused by the CAR T cells killing the tumor, and t is the clearance rate at which inflammation is removed. All parameters are assumed positive and

$$γ ( C ) = {γ}_{B} \frac{C}{{k}_{B} + C} .$$

The mathematical model and data from the Focused Cohort was integrated in the following way. For model fitting, the Julia packages DifferentialEquations, Optim, and BlackBoxOptim were used, which would find a set of best-fit parameters of equations (4) and (5) assuming previously established cancer cell proliferation rates of 0.15 per day. This provided a set of best-fit parameters for individual patients, describing the kinetics of ctDNA and tumor volume/inflammation according to equations (3)-(5). It was selected to present and discuss the fits for two representative patients based upon day 30 staging results: one PET+/ctDNA+ and one PET+/ctDNA−.

### Statistical Analysis

All correlations between MTV and ctDNA were performed by Spearman correlation and the coefficient (rs) was reported.

Median follow-up for survivors was calculated between the date of CAR T-cell administration and date of last contact; overall survival (OS) and progression free survival (PFS) from the time of CAR T-cell infusion until death or progression, or last contact. Differences in OS and PFS were found via Kaplan-Meier and log-rank test and hazard ratios (HR) and 95% confidence intervals (95% CI) reported. Survival was reported for the Focused Cohort at baseline per low versus high tumor burden group, defined by previously derived and validated baseline MTV cutoff value of 147.5 mL and pre-lymphodepletion ctDNA cutoff of 100 Lg/mL. Overall response (ORR), including partial (PR) and complete response (CR), at 3 and 6 months, as well as ORR and complete response if achieved by last follow-up were reported for the Entire Cohort. Cytokine release syndrome and CAR T-cell related encephalopathy syndrome rates were reported for the Entire Cohort.

P-value<0.05 was defined as statistically significant. Analysis was conducted using GraphPad Prism version 9.0.2 (161) for Windows (GraphPad Software, San Diego, California USA, www.graphpad.com)

### Results

### Patient Characteristics

Baseline patient characteristics and clinical outcomes for all patients are presented in Table 1. The median time between baseline ¹⁸F-FDG PET/CT and administration of CAR T-cell therapy was 33 (range, 1-137; interquartile range (IQR), 44.5) days.

TABLE 1
Patient Characteristics at the Time of Axi-
cel Infusion and Clinical Outcomes (n = 57).

| Characteristic | n = 57 (%) | |
|---|---|---|
| Age (years) | | |
| Median, range | 59, 19-76 | |
| Gender | | |
| Male | 34 | (60) |
| ECOG (0-5) | | |
| 0-1 | 54 | (95) |
| 2 | 3 | (5) |
| Histology | | |
| Diffuse large B-cell lymphoma (DLBCL), not | 32 | (56) |
| otherwise specified (NOS) | | |
| Unknown MYC and BCL2/BCL6 status | 4 | (7) |
| High grade B-cell lymphoma, with MYC and | 10 | (18) |
| BCL2 and/or BCL6 rearrangements | | |
| B-cell lymphoma, unclassifiable, with features | 11 | (19) |
| intermediate between DLBCL and classical | | |
| Hodgkin lymphoma | | |
| Unknown MYC and BCL2/BCL6 status | 3 | (5) |
| Primary mediastinal B-cell lymphoma | 4 | (7) |
| Stage (I-IV) | | |
| I/II | 15 | (26) |
| III/IV | 42 | (74) |
| LDH Level before conditioning > ULN | | |
| Yes | 37 | (65) |
| CRP Level before conditioning > ULN | | |
| Yes | 25 | (44) |
| Ferritin Level before conditioning > ULN | | |
| Yes | 31 | (54) |
| IPI score (1-5) | | |
| 0 | 4 | (7) |
| 1-2 | 23 | (40) |
| 3-5 | 25 | (44) |
| N/A or Primary mediastinal B-cell | 5 | (9) |
| lymphoma | | |
| Prior lines of therapy | | |
| Median, range | 3, 1-7 | |
| Bridging Therapy | | |
| Yes | 33 | (58) |
| Chemotherapy/targeted therapy | 15 | (26) |
| Steroids | 3 | (5) |
| Radiation therapy | 7 | (12) |
| Combination chemotherapy/targeted | 8 | (14) |
| therapy +/− steroids +/− radiation therapy | | |
| Received prior to baseline ¹⁸F-FDG | 10 | (18) |
| PET/CT | | |
| N/A | 1 | (2) |
| Outcome | n = 57 (%) | |
| Clinical Response to axi-cel | | |
| CR by last follow up | 35 | (61) |
| ORR by last follow up | 52 | (91) |
| ORR at 3 months | 31 | (54) |
| ORR at 6 months | 26 | (46) |
| Follow up and Survival | | |
| Median follow-up for survivors in months | 20.7 | (2.7-32.9) |
| (range) | | |
| Median OS in months (95% CIs) | Not reached | |
| Median PFS in months (95% CIs) | 13.4 | (12.2-13.5) |
| Toxicity, Grade | | |
| CRS 1-3 | 50 | (88) |
| 3 | 1 | (2) |
| N/A | 3 | (5) |
| CRES 1-4 | 30 | (53) |
| 3-4 | 15 | (26) |
| N/A | 3 | (5) |

### Biomarker Values and Correlation

Baseline and available post-treatment MTV and ctDNA values are reported in Table 2. The median processing time of a scan at baseline (n=42) was 30 min (range, 8-217 min, 84.5 min), at 1 month (n=47) 8 min (range, 2-180 min; IQR, 4 min), and at 3 months (n=35) 5 min (range, 2-145 min; IQR, 11 min). Processing time per scan correlated strongly with MTV at baseline (rs 0.71, P<0.0001), at 1 month (rs 0.81, P<0.0001), and at 3 months (rs 0.89, P<0.0001).

TABLE 2
ctDNA and MTV Values at Certain Timepoints Pre and Post Axi-cel.

Baseline

| Biomarker | n = 57 | n = 41 | 1 month | 3 months |
|---|---|---|---|---|
| MTV [mL] | Entire Cohort | Focused Cohort | n = 56* | n = 46† |
| Median | 52.93 | 42.68 | 0.74 | 0 |
| Range | 2.3-2256.24 | 2.3-2106.56 | 0-3369.13 | 0-697.97 |
| IQR | 283.3 | 130.24 | 12.06 | 15.73 [symbol] |
| ctDNA | n = 57‡ | n = 41 | | |
| [Lg/mL] | Entire Cohort | Focused Cohort | n = 54§ | n = 42∥ |
| Median | 95.09 | 58.1 | 0 | 0 |
| Range | 0-17903.02 | 0-6542.75 | 0-2944.85 | 0-10291.34 |
| IQR | 878.84 | 856.89 | 11.98 | 13.67 |

| | Day 0 | Day 7 | Day 14 | Day 21 | Day 56 | Day 180 | Day 270 | Day 300 | Day 360 |
|---|---|---|---|---|---|---|---|---|---|
| ctDNA | n = 43 | n = 57 | n = 54 | n = 49 | n = 35 | n = 26 | n = 12 | n = 1 | n = 20 |
| Median | 16.14 | 4.85 | 0 | 0 | 0 | 0 | 0 | 0 | 0 |
| Range | 0-12449 | 0-31664 | 0-14551 | 0-1540 | 0-8131 | 0-4916 | 0-1637 | 0-0 | 0-3180 |
| IQR | 342.2 | 101.8 | 28.06 | 30.6 | 7.850 | 0.1725 | 0 | 0 | 0 |

*Due to unavailable PET, MTV could not be derived for 1 patient not in CR and was marked 0 mL in 8 patients in CR
†Similarly, MTV could not be derived for 2 patients not in CR and marked as 0 mL in 10 patients in CR
‡ctDNA on day 0 was used as baseline in 1 patient without pre-lymphodepletion ctDNA
§ctDNA was not checked in 3 patients without disease progression
∥ctDNA was not checked in 11 patients without disease progression

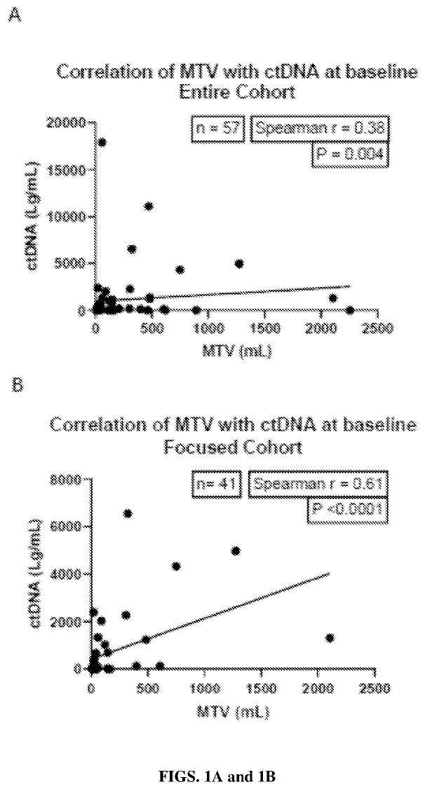

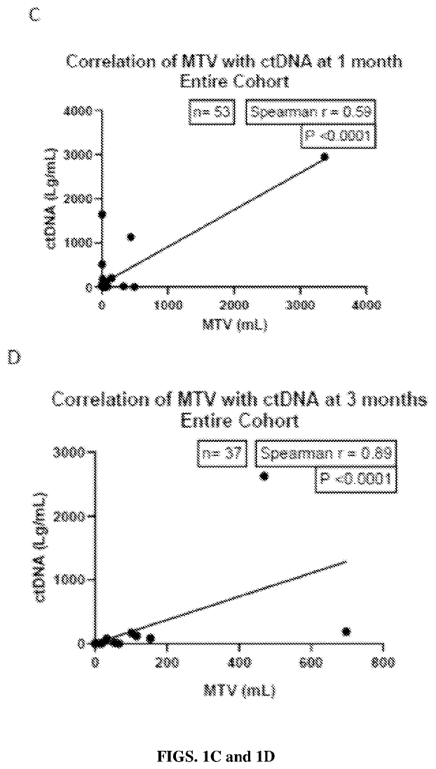

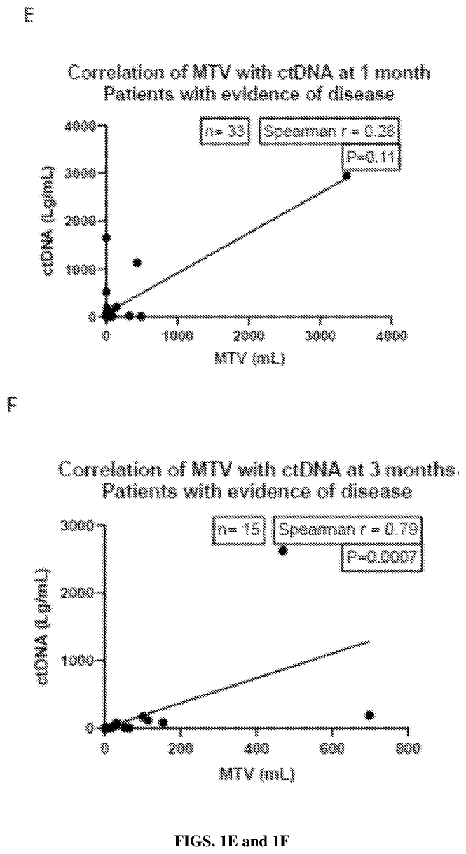

MTV and ctDNA values were correlated at baseline, 1 and 3 months post axi-cel. At baseline, in the Entire Cohort, MTV correlated weakly with ctDNA (rs 0.38, P=0.004); but in the Focused Cohort, the correlation became stronger (rs 0.61, P<0.0001) (FIGS. 1A and 1B). The impact of bridging therapy on measurements of baseline tumor burden was evaluated. For 23 patients who received bridging therapy after baseline imaging, there was no correlation of MTV and ctDNA (rs 0.08, P=0.71). However, for 34 patients who did not receive bridging therapy or received it prior to baseline imaging, there was significant correlation (rs 0.57, P=0.0004). Timing of the baseline scan in relation to axi-cel administration also impacted the relationship between MTV and ctDNA with significant correlation found when the scans were performed closer to therapy but not otherwise (quartile (Q) 1 rs 0.78, P=0.0008; Q2 rs 0.58, P=0.0296; Q3 rs 0.075, P=0.79; Q4 rs-0.066, P=0.82). At 1 month post CAR T-cell therapy, in the Entire Cohort (n=53), MTV had a moderate positive correlation with ctDNA (rs 0.59, P<0.0001) (FIG. 1C). At 3 months, in the Entire Cohort (n=37), the correlation between the two biomarkers was the strongest (rs 0.89, P<0.0001) (FIG. 1D). When patients with undetectable MTV and ctDNA were excluded, the correlation between the biomarkers became non-significant at 1 month (n=33) (rs 0.28, P=0.11) (FIG. 1E), but remained significant at 3 months (n=15) (rs 0.79, P=0.0007) (FIG. 1F).

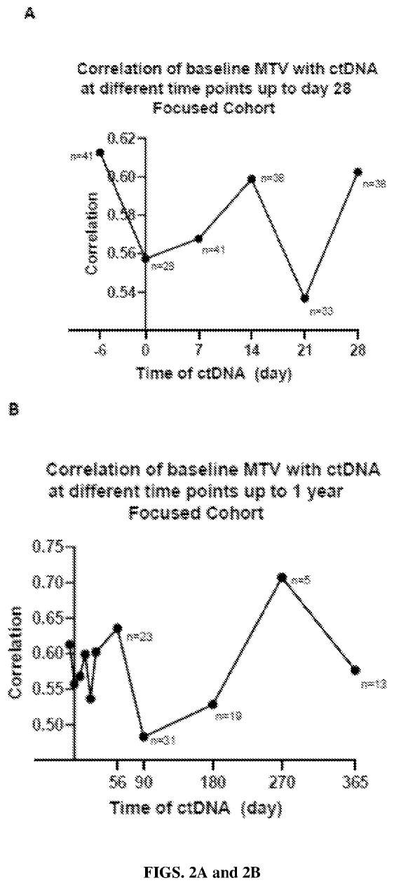

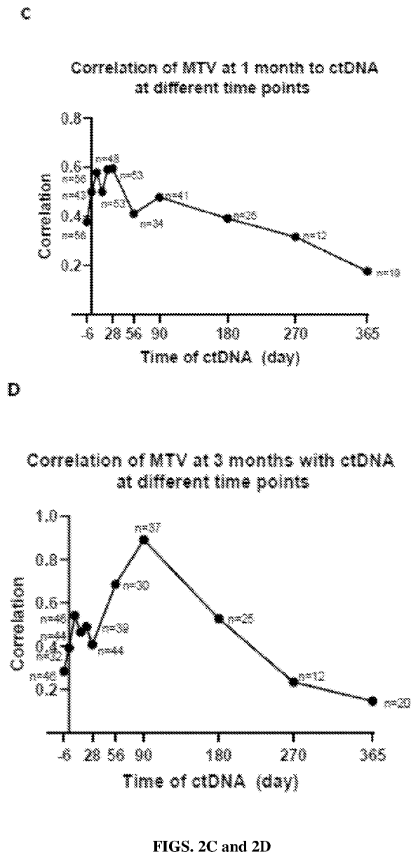

The relationship between MTV obtained at each of the three timepoints to all ctDNA values was further evaluated. In the Focused Cohort, baseline MTV correlated best with ctDNA at pre-lymphodepletion (rs 0.61, P<0.0001). The correlation varied more during the first month compared to later timepoints (FIG. 2A). The correlation after days 28 was overall poorer (FIG. 2B). In the Entire Cohort, MTV at 1 month correlated best with ctDNA on day 28 (n=53) (rs 0.60, P<0.0001) (FIG. 2C) and MTV at 3 months with ctDNA on day 90 (n=37) (rs 0.89, P<0.0001) (FIG. 2D).

Baseline Biomarker Association with Survival

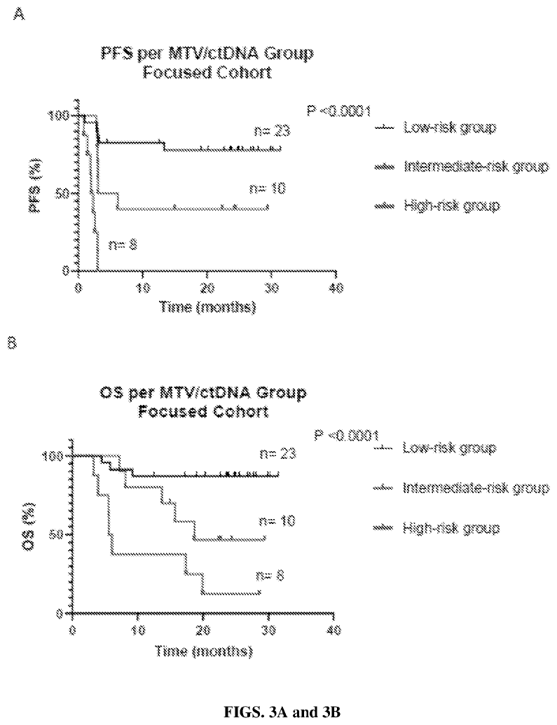

At baseline, in the Focused Cohort, 32 patients had low (range, 2.3-145.86 mL) and 9 patients high (range, 166.18-2106.56 mL) MTV; 24 patients had low (range, 0-98.16 Lg/mL) and 17 patients high (range, 127.92-6542.75 Lg/mL) ctDNA. Low compared to high MTV, and low compared to high ctDNA, were associated with better PFS (HR=0.17, 95% CI 0.03-0.71, P<0.0001; HR=0.11, 95% CI 0.04-0.3, P<0.0001) and OS (HR=0.19, 95% CI 0.04-0.8, P=0.0003; HR=0.13, 95% CI 0.04-0.39, P=0.0002), respectively. Patients with low MTV/low ctDNA (low-risk group) had better survival compared to those with either low MTV/high ctDNA or high MTV/low ctDNA (intermediate-risk group) or high MTV/high ctDNA (high-risk group) (FIG. 3).

### Patterns of Lymphoma Response and Progression

From baseline to 1 month, 51 patients had a confirmed decrease in MTV on imaging (n=56) with median reduction of MTV 47.78 mL (range, 1.91-2251 mL; IQR, 235.63 mL). The day 30 scan of one patient with PR was unavailable for review. This correlated to the Lugano response criteria, in which 4 patients progressed and 2 patients had stable disease (SD) (of which 1 had increased MTV and the other had a reduction). The scans of 19 patients were available for review at the time of progression: 15 patients had an increase of MTV>50% compared to prior scan, with half developing new lesions in addition to an increase in FDG uptake in a lesion.

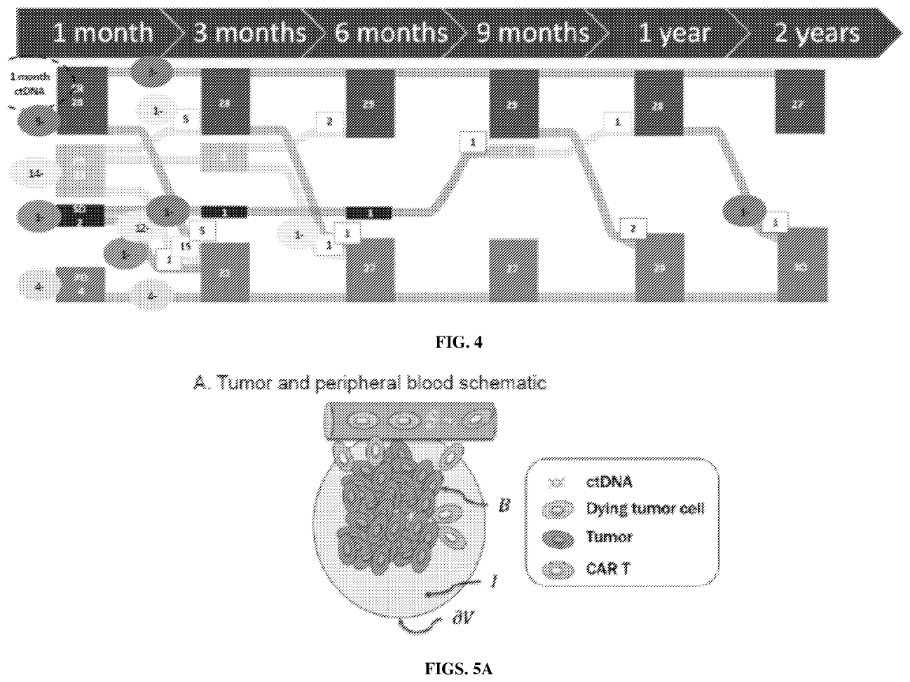

Responses to axi-cel are shown over time (FIG. 4). At 1 month post axi-cel, 28 patients were in CR: 5 patients had positive ctDNA with 2 patients experiencing progression of disease; 21 patients had negative ctDNA with 14 remaining in CR; 2 patients who remained in CR did not have ctDNA values available. At the same assessment time point, 25 patients were in PR: 14 patients had positive ctDNA with 13 whose disease progressed; 8 had negative ctDNA with 6 converting to CR; ctDNA was unavailable for 1 patient with progression of disease. There were 2 patients with SD: one who had positive ctDNA had progression of disease by 3 months and the other who had negative ctDNA achieved CR by 1 year. All 4 patients with progression of disease at 1 month had positive ctDNA.

For the patients with evidence of disease at 1 month (n=33), 19 had both detectable MTV and ctDNA with 18 experiencing progression of disease (4 at 1 month, 13 at 3 months, 1 at 6 months) and 1 achieving CR at 3 months. There were 9 patients with detectable MTV but negative ctDNA of which 7 achieved CR (1 marked as CR by Lugano criteria at 1 month, 3 at 3 months, 2 at 6 months, and 1 at 1 year), while 2 had progression of disease at 3 months. The remaining 5 of the 33 patients with undetectable MTV but positive ctDNA, 3 remained in CR, while 2 progressed (1 at 3 months, and 1 at 1 year).

Dynamics of Tumor Burden, ctDNA, and Inflammation

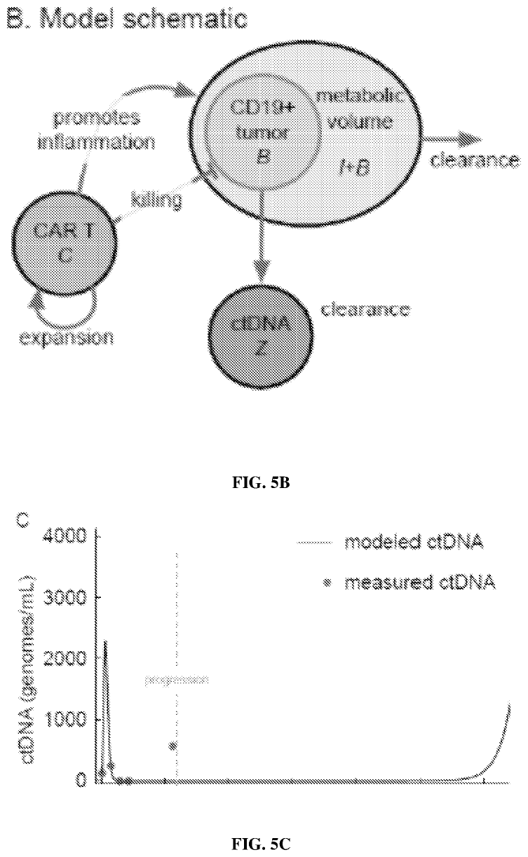

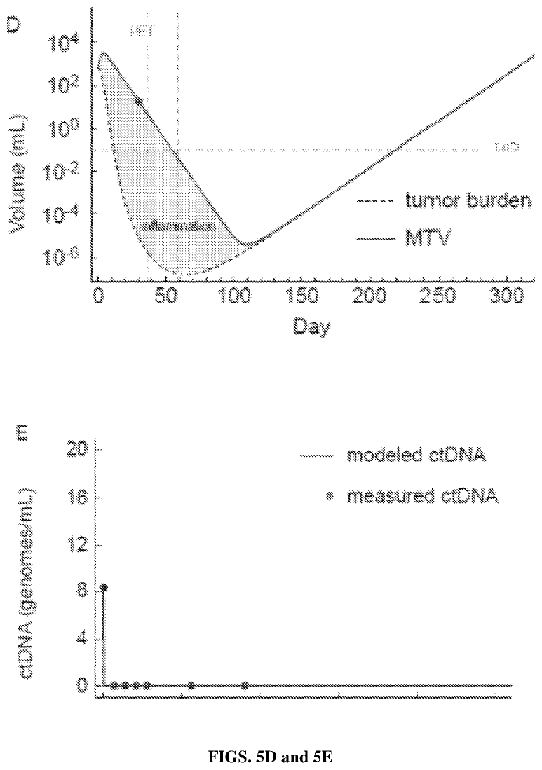

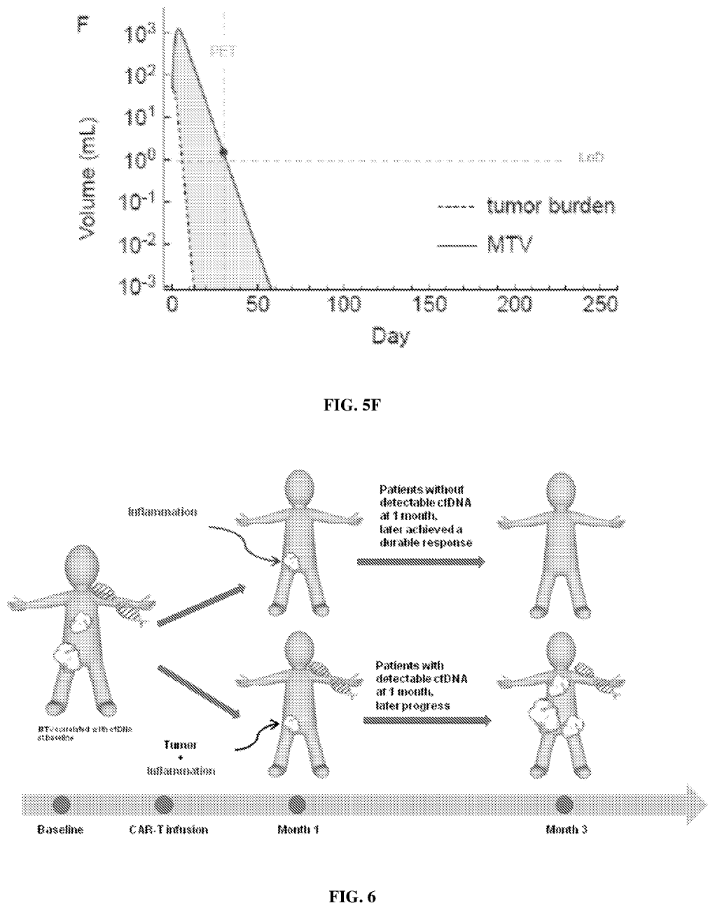

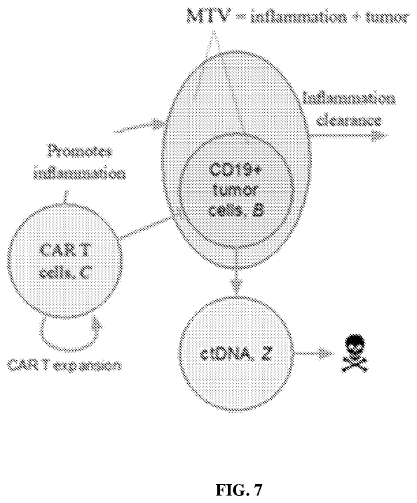

Given the uncertain response or durability of response at 1 month post CAR T-cell therapy based on imaging alone, a deterministic version of the mathematical model of CAR T cell dynamics was employed, expanded to include ctDNA and inflammation as part of the MTV measurement (FIG. 5A). It was contemplated that a portion of metabolic activity near or at the tumor site may be related to inflammation associated with the completed or ongoing killing of cancer cells by CAR T cells (FIG. 5B). The mathematical model was fitted to two patients with PET+ disease at day 30, and adequate longitudinal data to compare to the model (Methods): one patient who was PET+ and ctDNA+ at day 30 and later progressed, and one who was PET+ but ctDNA− at day 30, and achieved long term remission. Comparing these cases, the model recapitulated the dynamics of the first 30 to 60 days but showed some discrepancy at later time points (FIGS. 5C, 5D, 5E, and 5F), and correctly recapitulated progression (FIGS. 5C and 5D) and durable remission (FIGS. 5E and 5F).

### Discussion

The results of this multicenter, prospective sample study in patients with R/R LBCL receiving axi-cel demonstrated an ongoing treatment response in patients with non-progressing FDG-avid lesions on 1-month post-therapy ¹⁸F-FDG PET/CT scans. The lack of correlation between quantifiable MTV and detectable ctDNA implied activity, either continued cancer killing and/or inflammation, at known prior disease sites. Since patients had not undergone tissue biopsies, a modified version of an existing mathematical model of the dynamics of CAR T-cell treatment was employed at 1 month that successfully recapitulated clinical responses in patients with PR or SD. These results indicated that plasma ctDNA served as a physical biologic identifier of radiologically visualized, metabolically active, residual tumor. Given the test's low level of detection, its absence showed that FDG-avid lesions likely represented localized inflammation induced by CAR T-cell therapy. Thus, it was shown that concurrent ctDNA added specificity to the 1 month positive restaging ¹⁸F-FDG PET/CT.

This work focused on developing comprehensive non-invasive response assessments of patients undergoing CAR T-cell therapy. The imaged tumor's response was shown through the use of consecutively detected ctDNA via NGS. LBCL ctDNA monitoring has been shown to be a promising technique with prognostic approaches for accurate identification and quantification of disease at diagnosis, during and after first line therapy, pre as well as post CAR T-cell therapy. An alternative technique of low-pass whole-genome sequencing of cell-free DNA to find somatic copy number alterations has also been investigated in patients with LBCL treated with CAR T-cell therapy and in one study results at baseline were combined with surrogates of disease burden, lactate dehydrogenase and number of extranodal sites, to prognosticate clinical outcomes. Liquid biopsies can be particularly advantageous in cases where tissue biopsies are not feasible or the risks outweigh the benefits as in patients with PR or SD at 1 month after CAR T-cell therapy with small residual FDG-avid lesions on imaging.

By accurately and efficiently deriving MTV, the total amount of metabolic activity was quantified on imaging. This allowed for going beyond recognizing a scan result as a binary “positive” or “negative” for disease. Importantly, baseline MTV and ctDNA is concordant, particularly when removing the impacts of bridging therapy and timing. Both high baseline ctDNA and MTV were associated with poor clinical outcomes. This is a redemonstration of ctDNA's value as an emerging biomarker within a proportion (n=57) of the same cohort of patients previously reported (n=69) and another validation of MTV as an imaging biomarker in a largely unique patient cohort (n=42/57). Combining the two biomarkers, patients in the low-risk group pre axi-cel showed significantly better PFS and OS than those in the intermediate or high-risk groups. This prognostic model is incorporated into an ongoing clinical study (ClinicalTrials.gov Identifier: NCT05255354).

Herein, the lack of correlation between ctDNA and MTV at 1 month may be explained by the mechanism of action of CAR T-cell therapy and ongoing treatment response. Oscillations between the two variables during the first month after infusion resulted from active tumor killing induced directly or indirectly by axi-cel, which has a median time to radiologic response of 0.9 months. Death of tumor was confirmed by the reduction of median MTV and ctDNA in non-progressing patients at 1 month. While LBCL tumor typically consists of up to 90% tumor cells causing FDG uptake on scans, it was suspected that localized inflammation caused by the CAR T-cell therapy clearing tumor contributed to residual FDG-avid lesions on 1 month restaging ¹⁸F-FDG PET/CT. Loss of correlation at 1 month, but not 3 months, post therapy for those patients with either persistent MTV, ctDNA, or both was likely due to ongoing treatment response.

To reproduce tumor response at 1 month post axi-cel, a mathematical model was used. This modeling is an approach to conceptualize immunotherapy effects. A deterministic version of a hidden Markov model was used, a type of a stochastic method representing an autonomous system whose future state depends only on its current state at any given time. The hidden nature of the model comes from the fact that while MTV is observable, but is comprised not only of tumor but also inflammation. The difference between these two quantitates must be inferred. This inference can be accomplished by utilizing detectable ctDNA, which provides a proxy of tumor growth activity (vs. inflammation). Incorporating ctDNA in this study distinguishes it from other studies, where FDG-avidity on 1 month imaging alone was used to predict the risk of lymphoma progression. By depicting tumor dynamics, this model was able to predict clinical outcomes of patients with measurable MTV 1 month post axi-cel. The model supports localized tumor inflammation playing a role in ongoing tumor PET avidity, thus showing that in the absence of ctDNA, FDG-avid lesions on a scan may represent inflammation.

Serial plasma ctDNA measurements assisted with capturing the patients' ongoing response to therapy. The response is otherwise clearly visible only on spaced-out imaging studies. The clearance rate of ctDNA explains the remaining detectable ctDNA at 1 month in patients in CR by PET who eventually achieved durable CR. While ctDNA is undetectable in patients with CR at 1 month, upon relapse of disease, ctDNA rises sharply. In the original prospective study of the test, the dynamics of ctDNA were described in detail, and reported sensitivity of 94% and specificity of 82% for the test in the subjects with PR or SD. The results in this follow up study confirm that a one-time value of ctDNA reflects instantaneous tumor burden as evidenced by mismatched timing of measurement with MTV leads to poorer correlation.

This example includes the multicenter approach, the use of ctDNA, which relies on clonotype identification of tumors with a high specificity and sensitivity, the ability to quantify tumor burden accurately via MTV, the incorporation of data from multiple time points, and modification of an existing mathematical model as evidence for inflammation leading to PET positivity after CAR-T.

In conclusion, plasma ctDNA is shown to serve as a valuable supplementary test to standard ¹⁸F-FDG PET/CT scan imaging at 1 month by enhancing the accuracy of non-invasive response assessment of LBCL in patients receiving CAR T-cell therapy.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

### REFERENCES

Abramson J S, Palomba M L, Gordon L I, et al. Lisocabtagene maraleucel for patients with relapsed or refractory large B-cell lymphomas (TRANSCEND NHL 001): a multicentre seamless design study. Lancet. 2020 Sep. 19; 396(10254):839-852.
Al Zaki A, Feng L, Watson G, et al. Day 30 SUVmax predicts progression in patients with lymphoma achieving PR/SD after CAR T-cell therapy. Blood Adv. 2022 May 10; 6(9):2867-2871.
Barbany G, Arthur C, Liedén A, et al. Cell-free tumour DNA testing for early detection of cancer—a potential future tool. J Intern Med. 2019 August; 286(2):118-136.
Bekker R A, Zahid M U, Binning J M, et al. Rethinking the immunotherapy numbers game. Journal for ImmunoTherapy of Cancer. 2022; 10: e005107.
Benmebarek M R, Karches C H, Cadilha B L, Lesch S, Endres S, Kobold S. Killing mechanisms of chimeric antigen receptor (CAR) T cells. Int J Mol Sci. 2019 Mar. 14; 20(6):1283.
Cheng F, Su L, Qian C. Circulating tumor DNA: a promising biomarker in the liquid biopsy of cancer. Oncotarget. 2016 Jul. 26; 7(30):48832-48841.
Cherng H J, Sun R, Sugg B, et al. Risk assessment with low-pass whole-genome sequencing of cell-free DNA before CD19 CAR T-cell therapy for large B-cell lymphoma. Blood. 2022 Aug. 4; 140(5):504-515.
Cheson B D, Ansell S, Schwartz L, et al. Refinement of the Lugano Classification lymphoma response criteria in the era of immunomodulatory therapy. Blood. 2016 Nov. 24; 128(21):2489-2496.
Dean E A, Mhaskar R S, Lu H, et al. High metabolic tumor volume is associated with decreased efficacy of axicabtagene ciloleucel in large B-cell lymphoma. Blood Adv. 2020 Jul. 28; 4(14):3268-3276.
Frank M J, Hossain N M, Bukhari A, et al. Monitoring of circulating tumor DNA improves early relapse detection after axicabtagene ciloleucel in large B-cell lymphoma: results of a prospective multi-institutional trial. J Clin Oncol. 2021 Sep. 20; 39(27):3034-3043.
Goodman A M, Holden K A, Jeong A R, et al. Assessing CAR T-Cell therapy response using genome-wide sequencing of cell-free DNA in patients with B-cell lymphomas. Transplant Cell Ther. 2022 January; 28(1):30.e1-30.e7.
Hossain N M, Dahiya S, Le R, et al. Circulating tumor DNA assessment in patients with diffuse large B-cell lymphoma following CAR T-cell therapy. Leuk Lymphoma. 2019; 60(2):503-506.
Kimmel G J, Locke F L, Altrock P M. The roles of T cell competition and stochastic extinction events in chimeric antigen receptor T cell therapy. Proc Biol Sci. 2021 Mar. 31; 288(1947):20210229. Erratum in: Proc Biol Sci. 2022 Feb. 9; 289(1968):20212786.
Kuhnl A, Roddie C, Kirkwood A A, et al. Early FDG-PET response predicts CAR-T failure in large B-cell lymphoma. Blood Adv. 2022 Jan. 11; 6(1):321-326.
Kurtz D M, Jin M, Soo J, et al. Circulating tumor DNA is a reliable measure of tumor burden at diagnosis of diffuse large B cell lymphoma: an international reproducibility study. Blood. 2017; 130 (Supplement_1):310.
Locke F L, Ghobadi A, Jacobson C A, et al. Long-term safety and activity of axicabtagene ciloleucel in refractory large B-cell lymphoma (ZUMA-1): a single-arm, multicentre, phase 1-2 trial. Lancet Oncol. 2019; 20(1):31-42.
Meignan M, Hutchings M, Schwartz L H. Imaging in lymphoma: the key role of fluorodeoxyglucose-positron emission tomography. Oncologist. 2015 August; 20(8):890-5.
Roschewski M, Dunleavy K, Pittaluga S, et al. Circulating tumour DNA and CT monitoring in patients with untreated diffuse large B-cell lymphoma: a correlative biomarker study. Lancet Oncol. 2015 May; 16(5):541-9. Erratum in: Lancet Oncol. 2015 May; 16(5): e199.
Roschewski M, Rossi D, Kurtz D M, Alizadeh A A, Wilson W H. Circulating Tumor DNA in Lymphoma: Principles and Future Directions. Blood Cancer Discov. 2022 January; 3(1):5-15.
Schuster S J, Bishop M R, Tam C S, et al. Tisagenlecleucel in adult relapsed or refractory diffuse large B-cell lymphoma. N Engl J Med. 2019 Jan. 3; 380(1):45-56.
Yoon B J. Hidden Markov models and their applications in biological sequence analysis. Curr Genomics. 2009 September; 10(6):402-15.
Zhou L, Zhao H, Shao Y, et al. Serial surveillance by circulating tumor DNA profiling after chimeric antigen receptor T therapy for the guidance of r/r diffuse large B cell lymphoma precise treatment. J Cancer. 2021 Jul. 13; 12(18):5423-5431.

1. A method of treating cancer in a subject in need thereof, the method comprising:
isolating a tissue sample from the subject, wherein the tissue sample comprises circulating tumor DNA (ctDNA);
capturing an image from the subject with an imaging modality;
assaying the ctDNA in the tissue sample;
measuring metabolic tumor volume (MTV) from the image; and
wherein the presence of ctDNA and MTV in the subject or an increase in ctDNA and MTV in the subject relative to a control indicate the subject has a cancer; and
administering a treatment to the subject.

2. The method of claim 1, wherein the ctDNA is assayed with a DNA sequencing method.

3. (canceled)

4. The method of claim 1, wherein the imaging modality comprises a positron emission tomography (PET) scan.

5. The method of claim 1, wherein the tissue sample comprises a plasma sample.

6. The method of claim 1, wherein the treatment comprises a chimeric antigen receptor (CAR) T cell therapy.

7. (canceled)

8. (canceled)

9. The method of claim 1, wherein the cancer comprises a first tumor, a relapse tumor, or a refractory tumor.

10-19. (canceled)

20. A method of assessing efficacy of a chimeric antigen receptor (CAR) T cell therapy administered to a subject diagnosed with cancer, the method comprising:
isolating a tissue sample from the subject, wherein the tissue sample comprises circulating tumor DNA (ctDNA);
capturing an image from the subject with an imaging modality;
assaying the ctDNA in the tissue sample;
measuring metabolic tumor volume (MTV) from the image;
observing the subject for a level of inflammation; and
quantifying a correlation between the ctDNA, the MTV, the level of inflammation, and a concentration of CAR T cells, wherein efficacy of the CAR T cell therapy is ineffective when the ctDNA and MTV increases or remains unchanged relative to the concentration of CAR T cells, and wherein efficacy of the CAR T cell therapy is effective when ctDNA and MTV decreases relative to the concentration of CAR T cells.

21. The method of claim 20, wherein the correlation is quantified over a period of time.

22. he method of claim 20, wherein the method quantifies a rate of ctDNA degradation.

23. The method of claim 20, wherein the method quantifies a rate of tumor volume reduction, a rate of tumor volume growth, and a rate of tumor killing.

24. The method of claim 20, wherein the rate of tumor killing is proportionate to the concentration of CAR T cells.

25. The method of claim 20, wherein the method quantifies a rate of ctDNA moving from a cancer cell into peripheral blood.

26. The method of claim 20, wherein the method quantifies a rate of inflammation clearance.

27. The method of claim 20, wherein efficacy is assessed at least 30 days after the subject is administered the CAR T cell therapy.

28. (canceled)

29. (canceled)

30. (canceled)

31. The method of claim 20, wherein the ctDNA is measured one, two, or more times.

32. The method of claim 20, wherein the MTV is measured one, two, or more times.

33. The method of claim 20, wherein the level of inflammation is measured one, two, or more times.

34. (canceled)

35. (canceled)

36. The method of claim 20, wherein the ctDNA is obtained from a DNA sequencing method.

37. (canceled)

38. The method of claim 20, wherein the imaging modality comprises a positron emission tomography (PET) scan.

39. The method of claim 20, wherein the tissue sample comprises a plasma sample.

40. (canceled)
